(19) United States
(12) Patent Application Publication
(10) Pub. No.: US 2008/0089937 A1
(43) Pub. Date: Apr. 17, 2008

(54) Controlled release drug compositions and preparation methods

(75) Inventors: Yong Gan, Huairou (CN); Xinteng Zhou, Huairou (CN)

Correspondence Address: BEYER WEAVER LLP, P.O. BOX 70250, OAKLAND, CA, 94612-0250, US

(73) Assignee: Ocean Star International, Inc., Snowville, UT (US)

(21) Appl. No.: 11/599,150

(22) Filed: Nov. 13, 2006

(30) Foreign Application Priority Data
Oct. 13, 2006 (CN) 200610113725.7

(51) Int. Cl. A61K 9/24

(52) U.S. Cl. 424/473

(57) ABSTRACT

A drug core composition is described for enhancing controlled delivery of pharmaceutical active agents with low solubility in water. The drug core composition contains at least a drug-layer and a push-layer. The drug-layer contains at least a pharmaceutical active agent with low solubility in water and a hydrophilic polymer carrier. The push-layer contains at least osmopolymers, water-insoluble polymers and osmagents. An osmotic pump system containing the drug core composition is described wherein a semi-permeable membrane is coated outside of the drug core composition. The drug core composition provides drug release at a controlled rate particularly adaptable for release of therapeutic dosages over a period of 24 hours when administered once a day.

### 1. RELATED APPLICATION

Priority is claimed pursuant to 35 USC 119(a) from Chinese patent application Serial No. 200610113725.7, filed Oct. 13, 2006, incorporated by reference herein in its entirety.

### 2. FIELD OF INVENTION

The present invention relates to the field of pharmaceutical preparation, including controlled release drug core compositions, especially used for controlled release of drugs with low solubility, and controlled-release devices containing such compositions. Furthermore, the present invention provides methods for preparing drug core compositions comprising hydrophilic polymers, osmopolymers, insoluble polymers and osmagents.

### 3. BACKGROUND OF THE INVENTION

Many common preparations of drugs or other pharmaceutically active substances are sustained-release capsules, where the primary mechanism of release is through dissolution and/or diffusion of drug from the capsule. However, the bioavailability of the drug by this device is greatly affected by presence of food in the digestive tract. Typically, the time to the maximum concentration (Tₘₐₓ) in the bloodstream is reached by 4 to 5 hours under fasting conditions and by 6 to 7 hours when the capsules are administered with food. Taking the sustained-release capsules under fasting conditions results in about a 30 percent increase in bioavailability and from about 40 to 70 percent increase in peak concentrations (Cₘₐₓ) compared to conditions when taken with food. Due to the effect of food on sustained-release capsules, patients taking these capsules under fasting conditions may experience side effects from high blood concentration of the drug. Therefore, it is desirable to develop a new preparation for delivery of drugs which may avoid or alleviate the food effect of the sustained-release capsules, and which could also reduce the side effects when taken under fasting conditions.

With the existing technology of the present field, oral administration tablets (capsules) of osmotic pump and osmotic implant preparations are made based on osmotic pressure theory. Taking the differential osmotic pressure across the membrane wall as its power of drug delivery, an osmotic pump controlled-release tablet is one of the most useful controlled-release formulations. It can be designed to have an excellent zero order release profile to avoid being affected by pH of the environment, gastrointestinal peristalsis and food, and to show excellent in vivo and in vitro correlation. Delivery by an osmotic pump device typically avoids great fluctuations of blood concentration of the drug and reduces the frequency of administration, thereby increasing drug safety and effect, and probability of compliance by the patient.

An elementary osmotic pump (EOP) is the first generation product of the osmotic pump controlled-release tablets, which is suitable for the soluble drugs (solubility of which is usually from 5 to 30 g per 100 ml). Thus, the preparation technology of osmotic pump controlled release tablets still needs to be improved and simplified to make it suitable for a broader range of beneficial drugs including insoluble drugs. Also, new functional inert ingredients need to be found for substitution.

Push-pull osmotic pumps (PPOP) or multichamber osmotic pumps are used for controlled delivery of beneficial drugs based on the osmotic pressure theory. A PPOP typically utilizes a semi-permeable membrane-coated tablet having a drug-releasing orifice. However, it contains a two-layer or three-layer tablet core. One layer of the core, the drug layer, comprises the beneficial drug, hydrophilic polymers with osmotic activity and other ingredients. Another layer, the push layer, comprises water-soluble hydrophilic polymers, osmagents and other ingredients. The water-soluble hydrophilic polymers are t also called osmotic active polymers or osmopolymers, of which PEO (poly(ethylene oxide)) is a typical representative.

The tablet core is typically coated with a semi-permeable membrane. One or several orifices are drilled into the surface of the two sides of the membrane that are in communication with the drug layer and the exterior of the tablet for delivery of the drugs. After the osmotic pump tablet is put into fluid a condition, water enters through the orifice(s) and semi-permeable membrane into the tablet core and the drug is partially dissolved or suspended in water under the action of osmagents. The drug is released from the orifice(s) under the action of osmotic pressure. Meanwhile, the osmopolymers in the push layer absorb water to swell to push the drug, which aids the drug release. After dissolving in water, the osmopolymers also contribute to the maintenance of the osmotic pressure.

Commercially available products, such as Adalat® GITS (Bayer), Glucotrol® XL(Pfizer), use PEO as an ingredient. An osmotic pump device for controlled release of tamsulosin hydrochloride is disclosed in US 2005100602 and US2005100603. In a disclosed device, the tamsulosin salt is contained in a layer with a hydrophilic polymer carrier. The particularly preferred carrier is PEO. The drug core composition taking PEO as the major inert ingredient is suitable for controlled release of beneficial drugs with low solubility. However, we find that an osmotic pump controlled release tablet containing such a drug core composition using PEO as the major carrier also has some inherent disadvantages. Firstly, PEO can cause a rather extensive time lag in distributing the drug because of its slow speed of water absorption and hydration. The glass transition temperature (T_{g}) of PEO is typically in the range of 65° C. to 67° C. So, PEO is not ideally heat stable and accordingly can be problematic both in the preparation of the osmotic pump device and during storage. For example, it is difficult to remove solvent during the granulation process. Since the granulation temperature is not above 40° C., the residue of organic solvent would be high or it would take an abnormally long period of time to properly dry. During tablet pressing, the temperature will be increased because of friction. When the temperature is above 50° C., conglutinations may occur using PEO. Special equipment for cooling or slowing the compacting speed during tablet pressing becomes necessary. Similarly, the stored temperature of a PEO carrier must be relatively low in order to retain its ideal drug release characteristics. Thus, the storage of the devices will typically require careful temperature control.

Consequently, a new kind of drug core composition needs to be developed to facilitate the delivery method, preparation and device for controlled release of beneficial drugs with low solubility. Administering drug once a day is the preferred best mode. The preparation desirably should release the drug at a controlled rate at zero order release profile. Furthermore, the time lag of drug release of the formulation should be as short as possible, and the preparation and storage of said formulation should not be limited by temperature to the greatest extent.

### 4. SUMMARY OF THE DISCLOSURE

The present invention is directed to osmotic pump devices for administration of a biologically active substance at a controlled rate into a biological environment comprising: a core comprising a first layer (drug layer) containing a pharmaceutically effective amount of said low solubility biologically active substance and about 10 to 99 percent by weight of the first layer of hydrophilic polymer carrier, and a second layer (push layer) comprising about 10 to 80 percent by weight of the second layer of water-insoluble volume-swellable polymers, about 80 to 10 percent by weight of the second layer of water-soluble osmopolymers, and about 5 to 50 percent by weight of the second layer of osmagents.

The low solubility biologically active substance preferably has solubility in water of no greater than about 10 mg/ml. The ratio of weight of the first layer to the second layer is in the range of about 1:0.5 to 1:3.

A preferred hydrophilic polymer carrier comprises polyvinylpyrrolidone polymers and/or copolymers, such as a homopolymer of linear 1-vinyl-2-pyrrolidone groups or a copolymer of 1-vinyl-2-pyrrolidone and vinyl acetate in the mass proportion of about 1:10 to 10:1.

Useful classes of osmopolyers for the second layer include acrylic acid polymers, acrylic acid copolymers, hydroxypropyl cellulose, polyvinypyrrolidone polymers, polyvinylpyrrolidone copolymer and mixtures of two or more thereof. Useful acrylic acid polymers include homopolymers of acrylic acid, crosslinked with an allyl ether pentaerythritoil, allyl ether of sucrose, or allyl ether of propylene, crosslinked with an allyl ether pentaerythritoil, allyl ether of sucrose, or allyl ether of propylene.

Useful classes of water-insoluble volume-swellable polymers for the second layer include sodium starch glycolate, low-substituted hydroxypropyl cellulose, crosslinked carboxylmethyl cellulose sodium and mixtures of two or more thereof.

Useful classes of osmagents for the second layer include water-soluble inorganic salts, organic acids, saccharides and mixtures of two or more thereof. Salts include sodium chloride, potassium chloride, magnesium chloride, potassium sulphate, sodium sulphate and magnesium sulphate. Organic acids include ascorbic acid and tartaric acid. Saccharides include mannitol, sorbitol, xylitol, glucose and sucrose.

The first and second layers each may further comprise a lubricant, a glidant, and a colorant, such as magnesium stearic acid, silicon dioxide and mixtures of two or more thereof.

The device further comprises a wall surrounding the core comprising a semi-permeable material permeable to the passage of an exterior fluid and substantially impermeable to the passing of the biologically active substance. The semi-permeable material preferably comprises cellulose polymers, such as cellulose ethers, cellulose esters or cellulose ester ethers.

The device further comprises a passageway in the wall communicating with the first layer and the exterior of the device for delivery of the biologically active substance. The passageway typically has a diameter of about 0.2 to 1.2 mm.

Methods of preparing the core and the device are provided.

Useful PVP polymers and/or copolymers as carriers in the first layer have an average molecular weight in the range of about 5000-3,000,000.

### 5. BRIEF DESCRIPTION OF THE DRAWINGS

FIG. 1 Shows the drug release curves of tamsulosin hydrochloride from the sample prepared in Example 5 in four kinds of dissolution media, simulating the gastrointestinal tract under different conditions in vivo.

FIG. 2 shows the mean blood drug level-time curves of the glipizide controlled release tablet prepared in Example 10 and a commercial product.

FIG. 3 shows the logarithm of Mean Blood Drug Level-Time curves of the glipizide controlled release tablet prepared in Example 10 and a commercial product.

### 6. DESCRIPTION OF THE PREFERRED EMBODIMENTS

The present invention provides a controlled release osmotic pump device comprising a drug core composition. The controlled drug release of the drug core composition facilitates the aim of administering formulation containing the drug once a day.

The present invention further provides a formulation and preparing method for controlled release of beneficial drugs having low solubility during an extended period of time, thus allowing a 24-hour curative effect by administering drug once a day. The osmotic delivery of low solubility drugs according to the invention facilitates the release of beneficial drugs with an excellent zero order profile.

According to the present invention, a controlled release drug core composition suitable for low solubility beneficial drugs comprises at least a drug layer (first layer) and a push layer (second layer) wherein the drug layer comprises low solubility beneficial drug and hydrophilic polymer carriers and the push layer comprises at least osmopolymers, insoluble water-swellable polymers and osmagents. The osmopolymers include one of acrylic polymers and/or copolymers, hydroxypropyl cellulose, vinylpyrrolidone polymers and/or copolymers, or a mixture of two or more thereof.

The low solubility beneficial drugs contained in the drug layer according to the present invention are referred as to as drugs with water solubility of no more than about 10 mg per ml. This definition is intended to include the practically insoluble or insoluble drugs with the solubility of no more than 0.1 mg per ml, very slightly soluble drugs with the solubility of 0.1 to 1 mg per ml, and slightly soluble drugs with the solubility of 1 to 10 mg per ml.

The hydrophilic polymers utilized as carriers in the drug layer according to the present invention are preferably polyvinylpyrrolidone polymers and/or copolymers, which are about 10 to 99 percent, preferably 20 to 99 percent, by weight of the drug layer. The push layer preferably comprises about 10 to 80 percent, preferably 20 to 60 percent, by weight of the push layer of osmopolymers, about 10 to 80 percent, preferably 15 to 65 percent, by weight of the push layer of insoluble polymers and about 5 to 50 percent, preferably 5 to 30 percent, by weight of the push layer of osmagents. The osmopolymers are selected from the group consisting of acrylic polymers (including acrylic polymers and/or copolymers), hydroxypropyl cellulose, and polyvinylpyrrolidone polymers and/or copolymers, or a mixture of two or more thereof. The polyvinylpyrrolidone polymers and/or copolymers are preferable. The insoluble polymers preferably comprise one selected from the group consisting of sodium starch glycolate, low-substituted hydroxypropyl cellulose, crosslinked carboxylmethyl cellulose sodium, or a mixture of two or more thereof. The sodium starch glycolate is more preferred. The osmagents, also called osmotic active solutes, may be salts, acids and/or carbohydrates, such as sodium chloride, potassium chloride, magnesium chloride, sodium sulfate, potassium sulfate and/or magnesium sulfate. The acids may be ascorbic acid and/or tartaric acid and the carbohydrates may be mannitol, sorbitol, xylitol, glucose and/or sucrose.

The present invention will be described in terms of a two-layer osmotic pump device, typically, in the form of a tablet. The term “layer” is used for ease of description, but it is understood that a layer may be a compartment containing the described materials. One of the layers is the drug containing layer or drug layer containing the biologically active substance and a carrier as well as other ingredients as described herein. The other layer is the push layer which contains various kinds of osmopolymers, which are water-soluble hydrophilic polymers that, when dissolved, produce osmotic pressure in the drug layer. The push layer also contains water-insoluble volume-swellable polymers, which have high water absorption speed and high water absorption capacity so that they swell intensively when exposed to water. The swell of these water-insoluble polymers can have a mechanical push force on the drug release layer. Either or both of the drug layer and the push layer may contain an osmagent, also called an osmotic solute

The device will be surrounded by a wall of semi-permeable material. This material is permeable to the passage of water or body fluid such as aqueous fluids and other biological fluids, and substantially impermeable to the passage of the biologically active substance. Such semi-permeable materials include, but are not limited to cellulose polymers, such as cellulose ethers, cellulose esters, alkyl cellulose and cellulose ester ethers. It can be cellulose acetate, ethyl cellulose, cellulose diacetate, cellulose triacetate, etc, and preferably cellulose acetate.

There will be at least one passageway in the device, particularly in the form of a tablet, in the wall communicating with the layer containing the active ingredient and the exterior of the device in order to deliver the active ingredient from the device. Typically this is an orifice provided by drilling the tablet with a laser from the exterior to the first layer containing the active ingredient. The size of the orifice will in part determine the drug (active ingredient) release rate. The diameter of such an orifice in a typical device ranges from about 0.2 to 1.2 millimeters.

The present invention provides a two-layer or two-compartment osmotic pump device, preferably in the form of a tablet for delivering a biologically active substance to the gastrointestinal tract for absorption into the body. In general, the tablet will contain up to about 2 percent by weight of the biologically active substance based on the total weight of the tablet. In the drug-containing layer, the tablet will advantageously contain as a carrier for biologically active substance one or more PVP polymers and/or PVP copolymers, which comprise from about 10 to 99 percent by weight of the drug containing layer. The exact ratio of PVP polymer and/or PVP copolymer used within the drug layer is ultimately dependent upon the constituent of the drug core and the desired drug release character. A useful PVP polymer is Povidone, a synthetic homopolymer of linear 1-vinyl-2-pyrrolidone groups with a molecular weight in the range of about 5,000 to 3,000,000, typically about 1,300,000. The glass transition temperatures (T_{g}) of Povidone range from 130° C. to 176° C. depending upon the molecular weight. The T_{g} of Povidone K-90 (Plasdone K-90) is 174° C. A useful PVP copolymer is Copovidone, a copolymer of 1-vinyl-2-pyrrolidone and vinyl acetate in the mass proportion in the range of about 1:10 to 10:1. A typical mass proportion of Copovidone are 7:3, 3:2, 5:5 and 3:7, typically about 3:2. A typical molecular weight of Copovidone is about 50,000. Another Copovidone (Plasdone S-630) has a T_{g} of 105° C. Another useful PVP polymer is Crospovidone, a cross-linked 1-vinyl-2-pyrrolidone homopolymer. For preparations with polyvinylpyrrolidone polymers and/or copolymers as the main carrier, no special cooling conditions or storage conditions are required.

The drug-containing layer also typically contains a glidant, such as silicon dioxide, a lubricant such as magnesium stearic acid and a colorant, such as an inorganic colorant to distinguish it from the push layer.

The drug-containing layer may also contain other ingredients such as hydrophilic sustained-release materials, dilutors, adhesives, and solvents. These additional sustained-release materials may be, for example, water-soluble osmopolymers such as acrylic acid polymers or copolymers, such as Carbomers, hydroxypropylmethyl celluose (HPMC), and the like, or mixtures of two or more thereof. Commercially available Carbomers are typically homopolymers of acrylic acid, crosslinked with an allyl ether pentaerythritoil, allyl ether of sucrose, or allyl ether of propylene. The drug-containing layer may also contain osmagents such as sodium chloride, lactose, mannitol, glucose, sucrose, fructose or mixtures of two or more thereof.

The second (push) layer of the device contains from about 10 to 80 percent by weight of the push layer of water-soluble osmopolymers, which can adjust the release ratio of the drug from the device, such as acrylic acid polymers or copolymers, such as Carbomers, hydroxypropylmethyl cellulose (HPMC), and/or PVP polymers and/or copolymers such as Povidone and Copovidone.

The push layer also contains water-insoluble volume-swellable polymers, such as sodium starch glycolate, low-substituted hydroxypropyl cellulose, crosslinked carboxymethyl cellulose sodium, such as Croscarmellose sodium, and the like, or a mixture of two or more thereof. The water-insoluble polymers are usually used as disintegrants in common tablets, especially in rapid release preparations, wherein the excellent disintegrability of the insoluble polymers ensures the rapid disintegration and release of drugs. However, the action of rapid disintegration and release impedes the application of such insoluble polymers in sustained or controlled release formulations. The present invention successfully applies this kind of insoluble polymers in controlled release drug core compositions. While not intending to be bound by a particular theory, the mechanism of application of this kind of insoluble polymers in the present invention is believed to be due to the relatively large surface area and porosity characterizing such insoluble polymers in order to have an excellent water-absorbing rate and water-absorbing capacity. They swell rapidly and intensively after absorbing water. For example, sodium starch glycolate may enlarge in water by 300 times of its initial volume; the expansion rate of low-substituted hydroxypropyl cellulose is about from 500% to 700% (wherein the mass proportion of the substitution group is about 10% to 15%); and the crosslinked carboxylmethyl cellulose sodium may enlarge in water by 4 to 8 times of its initial volume. A preparation comprising of the drug core composition will absorb water quickly in the fluid environment in vivo, and its volume will swell and give a pushing act on the drug release.

The preferable dosage form is an osmotic pump system, and more preferably osmotic pump tablets. Particularly when the osmotic pump system comprises osmagents, the entry and absorption of water will be accelerated, and it will further promote the volume swelling speed and degree of swelling of the water-insoluble polymers. Thus the drug release time lag can be shortened and the drug release will be more complete.

The push layer may also contain a glidant, such as silicon dioxide, a lubricant such as magnesium stearic acid and a colorant to distinguish it from the drug layer.

The colorants for the drug layer and push layer may be any one of several members of colorants, including inorganic oxides such as red ferric oxide, yellow ferric oxide, purple ferric oxide, black ferric oxide, or mixtures thereof.

Plasticizers may be typical plasticizers such as diethyl phthalate, ethyl phthalate, triethyl citrate, polyethylene glycol (PEG), or mixtures thereof.

Light blockers may be materials such as titanium dioxide, talc, silicon dioxide, or mixtures thereof.

Pore formers may be materials such as glycerin, propylene glycol, polyethylene glycol, water soluble inorganic salts, or mixtures thereof.

Solvents used in the manufacture of the drug layers may be materials such as acetone, water, ethanol, methylene dichloride, methanol, isopropanol, or mixtures thereof.

The drug core composition is suitable for the delivery of pharmaceutically active agents with low solubility in water, such as drugs practically insoluble or insoluble in water with solubility of less than 0.1 mg/ml, drugs very slightly soluble in water with solubility of between 0.1 mg/ml and 1 mg/ml, and drugs slightly soluble in water with solubility of between 1 mg/ml and 10 mg/ml.

The drugs practically insoluble or insoluble in water can be, but not limited, Pyrimethamine, Acetyspiramycin, Dimethicone, Almitrine Bismesylate, Diflunisal, Selenium Sulfide, Dimercaptosuccinic Acid, Undecylenic Acid, Zinc Undecylenate, Testosterone Undecanoate, Hydrocortisone Butyrate, Triazolam, Magnesium Trisilicate, Adenosine Disodium Triphosphate, Soybean Oil, Diethylstilbestrol, Hydroxyprogesterone Caproate, Pentagastrin, Penfluridol, Bisacodyl, Benorilate, Subimed Sulfur, Altretamine, Salsaltate, Dihydroartemisinin, Pyrantel Pamoate, Diclofenamide, Dipyridamole, Dehydrocholic Acid, Estazolam, Lumefantrine, Protionamide, Beclometasone Dipropionate, Clobetasol Propionate, Testosterone Propionate, Probenecid, Levonorgesterl, Huperzine A, Bumetanide, Ibuprofen, Estradiol Valerate, Primidone, Carbamazepine, Carmustine, Carmofur, Folic Acid, Metildigoxin, Mefenamic Acid, Mebendzole, Tolbutamide, Trimethoprim, Metoclopramide, Methotrexate, Methyltestosterone, Cefdinir, Cefuroxime Axetil, Stanozolol, Sparfloxacin, Semustine, Nilestriol, Nimodipine, Nitrendipine, Serine, Mitomycin, Gemfibrozil, Diazepam, Digoxin, Dithranol, Dexamethasone, Danazol, Triamcinolone, Warfarin Sodium, Mifepristone, Noscapine, Ftivazide, Isotretinoin, Fenbufen, Bendrofluazide, Clarithromycin, Clotrimazole, Amphotericin B, Nitrofurantoin, Furosemide, Piroxicam, Praziquantel, Indapamide, Indometacin, Pindolol, Resrepine, Rifampicin, Albendazole, Azithromycin, Alfacalcidol, Alprazolam, Ciprofloxacin, Cyclosporin, Cyclandelate, Artmeisinin, Chlorambucil, Nandrolone Phenylpropionate, Estradiol Benzoate, Pizotifen, Lindance, Perphenazine, Felodipine, Fenofibrate, Absorbable Gelatin Sponge, Roxithromycin, Rotundine, Powdered Posterior Pituitary, Etacrynic Acid, Erythromycin Estolate, Etoposide, Etomidate, Enoxacin, Dactinomycin D, Ethisterone, Norgestrel, Norethisterone, Ethinylestradiol, Quinestrol, Famotidine, Prednisone, Bulleyaconitine A, Tamoxifen Citrate, Sulfasalazine, Halcinonide, Hydrocortisone, Aluminium Hydroxide, Hydrochlorothiazide, Heavy Magnesium Carbonate, Recombinant Human Insulin, Adipiodone, Meloxicam, Lomustine, Digitoxin, Fluphenazine Decanoate, Nicardipine Hydrochloride, Diphenoxylate Hydrochloride, Prazosin Hydrochloride, Amiodarone Hydrochloride, Cinnarizine, Glibenclamide, Gliclazide, Glipizide, Gliquidone, Zinc Oxide, Magnesium Oxide, Anrinone, Dapsone, Triamterene, Betamethasone, Insulin, Cystine, Progesterone, Naproxen, Raubasine, Ftibamzone, Phenolphthalein, Bismuth Aluminate, Liquid Paraffin, Tretinoin, Vitamin A, Vitamin B₂, Vitamin D₂, Vitamin D₃, Vitamin E, Vitamin K₁, Erythromycin Ethylsuccinate, Bifendate, Bifonazole, Chloramphenicol Palmitate, Erythromycin Stearate, Nitrazepam, Nifedipine, Tioguanine, Azathioprine, Barium Sulfate (Type I), Barium Sulfate (Type II), Sucralfate, Clofibrate, Clofazimine, Chlorotrianisene, Clonazepam, Niclosamide, Clozapine, Chlorprothixene, Clioquinol, Chlorpropamide, Chlortalidone, Oxazepam, Oxaprozin, Omeprazole, Sulpiride, Sulindac, Probucol, Ferrous Fumarate, Artemether, Ketoprofen, Ketoconazole, Iodine, Iodinated Oil, Iophendylate, Iopanoic Acid, Bismuth Subcarbonate, Calcium Carbonate, Estradiol, Acedapsone, Desoxycortone Acetate, Cortisone Acetate, Megestrol Acetate, Menadiol Diacetate, Medroxyprogesterone Acetate, Dexamethasone Acetate, Triamcinolone Acetonide Acetate, Prednisone Acetate, Prednisolone Acetate, Fluocinonide, Fludrocortisone Acetate, Hydrocortisone Acetate, Chlormadinone Acetate, Sulfamethoxazole, Sulfadoxine, Sulfafurazole, Sulfadiazine, Sulfadiazine Silver, Sulfadiazine Zinc, Calcium Hydrogen Phosphate, etc.

The drugs very slightly soluble in water can be, but not limited, Acetazolamide, Proglumide, Propylthiouracil, Cefoperazone, Kitasamycin, Griseofulvin, Sultamicillin Tosilate, Benzoyl Peroxide, Triamcinolone Acetonide, Amobarbital, Erythromycin, Meleumycin, Benzathine Benzylpenicillin, Pipemidic Acid, Allopurinol, Diatrizoic Acid, Aciclovir, Phenobarbital, Benzocaine, Fenoprofen Calcium, Adrenaline, Prednisolone, Theophylline, Piperacilin, Fleroxacin, Haloperidol, Buprenorphine Hydrochloride, Flunarizine Hydrochloride, Meclozine Hydrochloride, Phenoxybenzamine Hydrochloride, Propafenone Hydrochloride, Bromhexine Hydrochloride, Aminoglutethimide, Norfloxacin, Phenolsulfonphthalei, Econazole Nitrate, Clemastine Fumarate, Mercaptopurine, Tyrosine, Carbocisteine, Camphor, etc.

The drugs slightly soluble in water can be, but not limited, Tamsulosin Hydrochloride, Dried Calcium Sulfate, Aspartic Acid, Ubenimex, Salicylic Acid, Deslanoside, Levodopa, Carbimazole, Carbidopa, Carboprost Methylate Thiamphenicol, Metronidazole, Busulfan, Caftazidime, Cefaclor, Cefalexin, Cefadroxil, Cimetidine, Tropicamide, Moclobemide, Tryptophan, Minoxidil, Isocarboxazid, Crotamiton, Ganciclovir, Glutamic Acid, Aspirin, Amoxicillin, Atenolol, piperazine Ferulate, Phenylpropanol, Benzoic Acid, Dimenhydrinate, Zinc Citrate, Clomifene Citrate, Fluconazole, Halothane, Cisplatin, Alprostadil, Berberine Hydrochloride, Maprotiline Hydrochloride, Amiloride Hydrochloride, Benzhexol Hydrochloride, Chlortetracycline Hydrochloride, Loperamide Hydrochloride, Cyproheptadine Hydrochloride, Oxprenolol, Ampicillin, Homoharringtonine, Chloramphenicol Succinate, Tinidazole, Puerarin, Isosorbide Dinitrate, Dihydralazine Sulfate, Quinidine Sulfate, Chlordiazepoxide, Chloramphenicol, Procaine Benzylpenicillin, Ketotifen Fumarate, Iotalamic Acid, Idoxuridine, Lithium Carbonate, Zinc Acexamate, Chlorhexidine Acetate, Tiabendazole, Piperaquine Phosphate, Fosfomycin Calcium, Fosfomycin Sodium, Spironolactone, etc.

Methods of making osmotic pump devices are known in the art such as described by Santus et al., J of Controlled Release, 35, 1-21 (1995); and U.S. Pat. No. 4,765,989, the contents of which are incorporated by reference into the present specification.

Although examples of sustained or controlled drug release dosage forms comprising the drug core composition are described herein, and the preparation method and the administration method of the dosage forms all relate to oral osmotic dosage form, they should not be considered as limiting the scope of the invention in any way. For example, the osmotic dosage form can be of two-layer or three-layer osmotic pump system form, and preferably two-layer osmotic pump system. It can be a tablet form or a capsule form, and preferably a tablet form.

The ratio of the drug-layer to the push-layer in the drug core composition should be 1:0.5-3, and preferably 1:0.5-1.5, more preferably 1:0.8-1.2, and most preferably 1:1.

A preparation method of the controlled release drug core composition comprising low soluble pharmaceutical active agents is also provided. The method comprises: (1) the preparation of the drug-layer: first all the ingredients are sieved through a 60-mesh sieve, and then the pharmaceutically active agent is evenly mixed with the ingredients, and the alcohol solution with concentration, typically of no less than 40%, is sprayed in and they are granulated; (2) the preparation of the push-layer: first all the ingredients are sieved through a 60-mesh sieve, and then the osmopolymers, water-insoluble polymers and osmagents are evenly mixed, and alcohol solution, typically with concentration of no less than 40%, is sprayed in and they are granulated; and (3) the drug-layer is pressed into form first and then the granules of the push-layer are added and they are pressed into a drug core. The drug core composition has at least one drug-layer and one push-layer.

The ingredients in the drug-layer contain magnesium stearic acid, colorants, polyvinylpyrrolidone polymers and/or copolymers, osmagents and/or silicon dioxide, and the ingredients in the push-layer still contains adhesives, colorants and/or glidants.

When the dose of the pharmaceutical active agents is very low, the preparation method of the drug-layer may be as follows. All the ingredients are first mixed, then the pharmaceutically active agent is dissolved into the alcohol solution, and sprayed into the mixed ingredients, followed by granulation.

A method of preparing an osmotic pump system comprising of the drug core composition is provided. The drug core composition is coated with a semi-permeable membrane and then dried. The coating media may comprise acetone, water, alcohol, dichloromethane, methanol, and/or isopropyl alcohol, or a mixture of two or more thereof.

The pump system may be coated with an anti-damp film coat over the semi-permeable membrane.

A preferred preparation method of the osmotic pump system is as follows.

First, prepare the drug core composition:

The preparation of the drug-layer: All the ingredients are sieved through a 60-mesh sieve, and then the pharmaceutically active agent is evenly mixed with the ingredients mentioned above (such as colorants, for example, yellow ferric oxide, polyvinylpyrrolidone polymers and/or copolymers, some other kinds of osmopolymers, osmagents, and silicon dioxide acting as glidant). The mixture is added into a fluid-bed, and is sprayed with 40˜100% alcohol water solution, granulated and dried. Magnesium stearic acid is added and evenly mixed.

The preparation of the push-layer: All the ingredients are sieved through a 60-mesh sieve, and then the osmopolymers, water-insoluble polymers and osmagents are mixed evenly, preferably with adhesive, colorant and silicon dioxide acting as glidant. The mixture is added into a fluid-bed and alcohol solution with concentration of no less than 40% is sprayed in and the mixture is granulated and then dried. Magnesium stearic acid is added and evenly mixed.

The alcohol concentration in water solution mentioned above is preferably 60˜95%, and more preferably 75˜95%.

Then an osmotic pump system is prepared, taking a tablet form for example:

Press one layer of the drug core composition, such as the drug-layer, into form, and then add the other layer, such as the push-layer, and press into a two-layer tablet core. Preferably coat the drug core composition with a semi-permeable membrane and dry it. Then drill a passageway by machine or laser into the semi-permeable membrane adjacent to the drug-layer. An anti-damp film coat is coated preferably over the semi-permeable membrane, and it is dried. The coating media of the semi-permeable membrane when coated may be selected from acetone, water, alcohol, dichloride, methanol, and isopropylalcohol, or their mixture of two or more thereof. It is preferably acetone. For example, cellulose acetate (or other cellulose derivative) and/or diethyl phthalate (DEP) is dissolved into acetone to form the coating solution and the membrane is coated using a coating machine. The anti-damp film coat may improve the appearance of the preparations and it can also provide color marking.

The technology of the passageway drilling into the membrane or film coating is known in the pharmaceutical field.

The osmotic pump system has many advantages. The drug can be released at a constant rate over an extended time period. The drug release is not significantly affected by the pH value of the medium, the creepage of the GI tract, the food taken, etc. There is good in vitro and in vivo correlation. There is no large fluctuation phenomenon of plasma drug concentration, which is common for the usual oral dosage forms. The drug administration times can be deduced, and the safety, efficiency and the compliance is improved largely. Furthermore, the use of polyvinylpyrrolidone polymers and/or copolymers instead of PEO and the use of water-insoluble polymers can further enhance the advantage of the osmotic pump system. The time lag of the drug release can be shortened and the drug release may be more complete. The system is more suitable for the controlled delivery of low soluble pharmaceutically active agents.

The daily dosage of the biologically active substance may be determined on a case-by-case basis keeping within an amount that is pharmaceutically effective for therapy or prevention of a particular disease or condition. Hence, tablets containing such amounts designed to deliver the payload of the desired daily dosage of the drug within 24 hours by sustained release would suffice. Typical biologically active substances useful in accordance with the invention include, but are not limited to, substances that can be delivered from the device to produce a beneficial and useful therapeutic result in the host, such as physiologically or pharmacologically active substances that produce a local or systemic effect in the host. Exemplary active substances include anti-inflammatories, antimicrobials, antihypertensives, cardiovascular drugs, diuretics, hormonals, hyperglycemics, muscle relaxants or contractants, sedatives, urinary tract drugs, vitamins, polypeptide drugs, and the like.

The following examples are illustrative of the present invention, but they should not be considered as limiting the scope of the invention in any way.

### EXAMPLE 1

Prescription:
(1) Drug layer (per tablet):

| | | |
|---|---|---|
| Nifedipine | 33 | mg |
| Povidone (Plasdone K-90D) | 30 | mg |
| Copovidone (Plasdone S630) | 91 | mg |
| Magnesium stearate | 1.5 | mg |
| Silicon dioxide | 0.5 | mg |

(2) Push layer (per tablet):

| | | |
|---|---|---|
| Sodium starch glycolate | 37 | mg |
| HPMC (K15M) | 30 | mg |
| Carbomer (971PNF) | 8 | mg |
| Sodium Chloride | 21 | mg |
| Copovidone (Plasdone S630) | 15 | mg |
| Red ferric oxide | 1.1 | mg |
| Magnesium stearate | 0.6 | mg |
| Silicon dioxide | 0.4 | mg |

(3) Semipermeable Membrane (Amount used per 1000 tablets)

| | | |
|---|---|---|
| Cellulose acetate | 59.5 | g |
| Diethyl phthalate | 3 | g |
| Acetone | 1500 | ml |
| Weight of the semi-permeable membrane | 38 | mg |

(4) film coat:

Spectrablend ™ Pink (CM-0317)

Preparation Method

1. Preparation of the Drug-Layer Granules:

Operating in the dark or under golden fluorescent or other low-actinic light. Nifedipine, Povidone (Plasdone K-90) and Copovidone (Plasdone S630) are sieved through a sieve of 60 mesh and homogeneously mixed together with silicon dioxide. Then the solid mixture is added into a fluid bed granulator, and a pre-prepared alcohol solution is sprayed to affect granulation. Water residue percent, drug content percent, content uniformity and related compounds are determined after drying. Then magnesium stearate is added and mixed. Granules of drug layer are formed.

2. Preparation of the Push-Layer Granules:

First, all the ingredients are sieved through a sieve of 60 mesh separately, then Sodium CMS, HPMC, Carbomer, NaCl, Copovidone (Plasdone S630), red ferric oxide are mixed together with silicon dioxide. The mixture is added into a fluid-bed granulator, then alcohol water solution is sprayed to make granulation. Drying, then water residue percent is determined. Then magnesium stearate is added and mixed. Granules of push layer are formed.

3. Tablet Pressing:

Operating in the dark or under golden fluorescent or other low-actinic light. The two-layer tablet cores are compacted with the two types of granules. The diameter is 8 mm. Drug hardness and content percent and content uniformity of the two-layer tablets are determined.

4. Tablet Coating with Semi-Permeable Membrane:

The semi-permeable membrane is coated outside of the tablet core then dried at 45° C. for about 3 hours. The weight gain of tablet is controlled carefully during the coating process. The organic solvent residue amount is determined.

5. A pore with diameter of 0.9 mm is drilled in the wall adjacent to the drug-layer by machine or laser. The drug release profile is measured.

6. Tablet Coating with Moisture-Proof Film Coat:

The tablet after drilling is coated by the moisture-proof film coat then dried at 45° C. for about 3 hours. Then quality analysis is conducted fully, including the drug content percent, drug content uniformity, drug release profiles, related compounds, residue amount of acetone, and others.

Povidone(Plasdine K-90D) may be changed by a different amount of Plasdone K-90 or Plasdone K-15 or Plasdone K-30 or Plasdone K-60 or Plasdone K-120 or mixtures thereof. HPMC (K15M) may be changed by a different amount of HPMC K4M or HPMC K100M or HPMC K100LV or mixtures thereof.

### EXAMPLE 2

Under the condition of different pH value mediums, the drug release of tablet from Example 1 (test product) and commercial product (Adalat GITS, made by German Bayer Inc.) is tested separately. By usage, the dosage is 110%, for example, the theoretical value is 30 mg, but in practice the dosage is 33 mg.

The results of drug release are shown in Table 1.

(1) Chlorhydric acid solution with 1% sodium dodecylsulfate (pH1.2);

(2) Acetic acid-sodium acetate buffer solution with 1% sodium dodecylsulfate (pH4.5);

(3) Phosphate-citron acid buffer solution with 1% sodium dodecylsulfate (pH6.8).

TABLE 1
The drug release of test product and commercial product

| | | Cumulated drug release (%) | | | | |
|---|---|---|---|---|---|---|
| Drug release medium | | 4 h | 8 h | 12 h | 16 h | 24 h |
| Commercial | pH 1.2 | 8.2 | 28.6 | 48.7 | 62.8 | 95.1 |
| product | pH 4.5 | 8.9 | 25.8 | 44.2 | 63.5 | 93.5 |
| | pH 6.8 | 9.1 | 27.4 | 46.6 | 66.0 | 98.8 |
| Test product | pH 1.2 | 10.5 | 30.7 | 50.5 | 65.4 | 96.2 |
| | pH 4.5 | 9.2 | 29.9 | 50.5 | 67.9 | 96.9 |
| | pH 6.8 | 10.1 | 32.3 | 55.3 | 73.3 | 103.8 |

The drug release of the test product from Example 1 and the commercial product both meet the requirement of provided a useful dosage over 24 hours. But compared with the commercial product, the test product has a less time lag and is more fully released.

### EXAMPLE 3

Prescription:
(1) Drug layer (per tablet):

| | | |
|---|---|---|
| Nifedipine | 33 | mg |
| Povidone (Plasdone K-90D) | 30 | mg |
| Copovidone (Plasdone S630) | 91 | mg |
| Magnesium stearate | 1.5 | mg |
| Silicon dioxide | 0.5 | mg |

(2) Push layer (per tablet):

| | | |
|---|---|---|
| L-HPC | 150 | mg |
| HPMC (K15M) | 30 | mg |
| Carbomer (971PNF) | 10 | mg |
| Sodium chloride | 33 | mg |
| Copovidone (Plasdone S630) | 30 | mg |
| Red iron oxide | 1.1 | mg |
| Magnesium stearate | 0.6 | mg |
| Silicon dioxide | 0.4 | mg |

(3) Semipermeable Membrane (Amount used per 1000 tablets)

| | | |
|---|---|---|
| Cellulose acetate | 59.5 | g |
| Diethyl phthalate | 3 | g |
| Acetone | 1500 | ml |
| Weigh of the semi-permeable membrane | 38 | mg |

(4) film coat:

Spectrablend ™ Pink (CM-0317)

Preparation method as Example 1.

### EXAMPLE 4

Except that L-HPC is changed by croscarmellose sodium, prescription and preparation method as Example 3.

### EXAMPLE 5

(1) Drug layer (per tablet):

| | | |
|---|---|---|
| Tamsulosin hydrochloride | 0.2 | mg |
| Copovidone (Plasdone S630) | 100 | mg |
| Yellow iron oxide | 0.07 | mg |
| Magnesium stearate | 1 | mg |
| Silicon dioxide | 0.5 | mg |

(2) Push layer (per tablet):

| | | |
|---|---|---|
| Sodium starch glycolate | 32 | mg |
| HPMC | 14 | mg |
| Carbomer | 5.7 | mg |
| Sodium chloride | 24 | mg |
| Copovidone (Plasdone S630) | 18 | mg |
| Red iron oxide | 1 | mg |
| Magnesium stearate | 0.5 | mg |
| Silicon dioxide | 0.4 | mg |

(3) Semipermeable Membrane (per tablet)

| | | |
|---|---|---|
| Cellulose acetate | 20 | mg |

(4) film coat (per tablet)

| | | |
|---|---|---|
| Spectrablend ™ (CM 0165) | 17 | mg |

Preparation Method

1. Preparation of the Drug-Layer Granules:

Yellow ferric oxide, and Copovidone (Plasdone S630) are sieved through a sieve of 60 mesh and homogeneously mix together with silicon dioxide. Then the solid mixture is added into a fluid bed granulator, and a pre-prepared alcohol solution containing tamsulosin hydrochloride is sprayed to affect granulation. Water residue percent, drug content percent, content uniformity and related compounds are determined after drying. Then magnesium stearate is added and mixed. Granules of drug layer are formed.

2. Preparation of the Push-Layer Granules:

First, all the ingredients are sieved through a sieve of 60 mesh separately, then Sodium CMS, HPMC, Carbomer, NaCl, Copovidone (Plasdone S630), red ferric oxide are mixed together with silicon dioxide. The mixture is added into a fluid-bed granulator, then alcohol water solution is sprayed to granulate. Drying, then water residue percent is determined. Then magnesium stearate is added and mixed. Granules of push layer are formed.

3. Tablet Pressing:

The two-layer tablet cores are compacted with the two types of granules. The diameter is 8 mm. Drug hardness and content percent and content uniformity of the two-layer tablets are determined.

4. Tablet Coating with Semi-Permeable Membrane:

The semi-permeable membrane is coated outside of the tablet core then dried at 45° C. for about 24 hours. The weight gain of tablet is controlled carefully during the coating process. The organic solvent residue amount is determined.

5. A pore with diameter of 0.9 mm is drilled in the wall adjacent to the drug-layer by machine or laser. The drug release profile is measured.

6. Tablet coating with moisture-proof film coat, then dried at 45° C. for about 12 hours.

Test method of the drug release as follow:

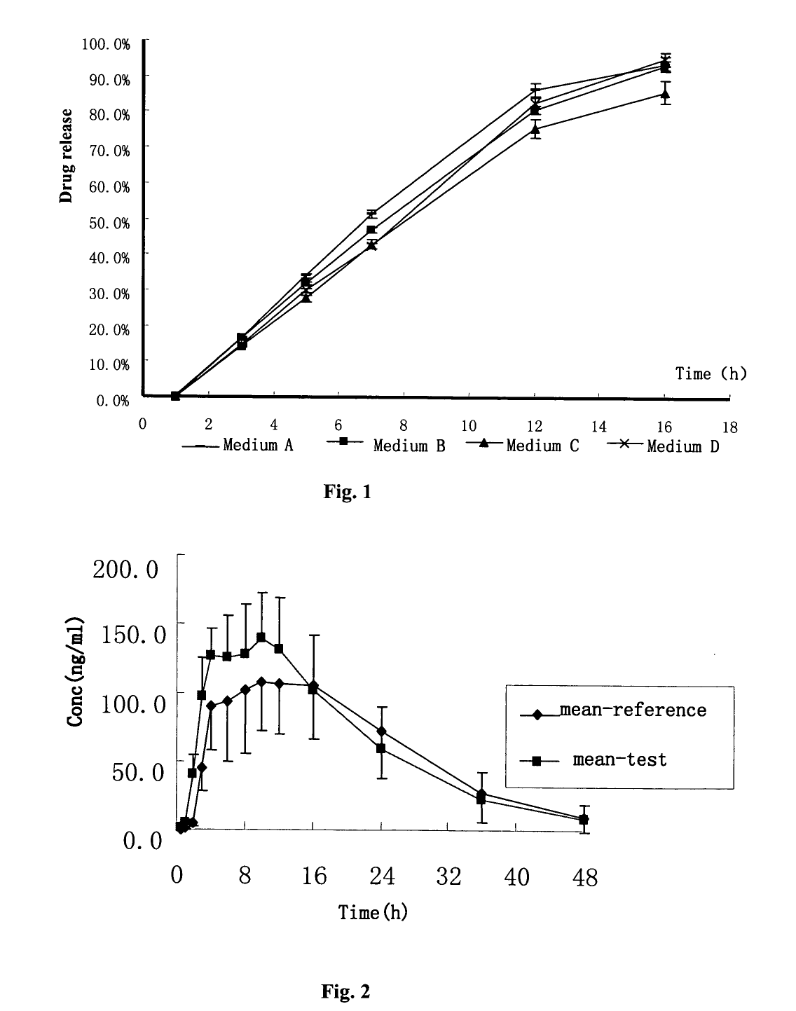

The drug release profile is measured in a type 2 dissolution apparatus (paddle) according to the China Pharmacopeia at 37° C. in 500 ml water at 50 rotations per minute. At 3, 5, 7, 12, and 16 hours, 10-ml are sampled respectively and the same volume of new dissolution media is immediately added. The samples are filtered with 0.45-μm micropore filter membrane as test solution. A portion of tamsulosin hydrochloride reference substance is dried at 105° C. for 2 hours, dissolved in water-acetonitrile solution (65:35) and diluted to obtain a solution having a known concentration of 0.5 mg per mL. A portion of this solution is diluted with water to obtain a solution having a known concentration of 0.4 μg/mL as reference solution. Then 80 μl of test and reference solutions are respectively injected into the chromatograph to determine the release of drug at the different times for each tablet. The detection chromatograph system is as follows: the column is ODS packed, the mobile phase is acetonitrile-perchloric acid solution (Dissolve 8.7 ml perchloric acid and 3 g NaOH into 1900 mL water, adjust it with NaOH solution to the value of pH equal to 2.0, then add water to the full scale of 2000 ml) (35:65, v/v). The flow rate is 1.0 ml/min. The wavelength of determination is 225 nm. Using sample above, the gastrointestinal tract environment in vivo is simulated with the four media above-mentioned and the effects of food, pH and gastrointestinal peristalsis on the drug release are evaluated. The results of release are shown in FIG. 1.

Results of the drug release: The cumulated drug release at 1, 3, 5, 7, 12 and 16 hour is 10%, 33%, 53%, 76%, 93% and 96%.

### Preparation of Simulated GI Media

Four kinds of dissolution medium simulating the gastrointestinal tract environment are designed to evaluate the effect of gastrointestinal peristalsis, pH and food on the drug releasing property.

Medium A (SGF, simulated gastric fluid without pepsin):pH 1.2

| | |
|---|---|
| HCl | 7.0 mL |
| NaCl | 2.0 g |
| Water | sufficient |
| Total volume | 1000 mL |

### Medium B (SIF, simulated intestinal fluid without pancreatin):pH 6.8

| | |
|---|---|
| KH₂PO₄ | 6.8 g |
| NaOH | 0.944 g |
| Water | sufficient |
| Total volume | 1000 mL |

### Medium C (FaSSIF, simulated intestinal fluid, fasted condition):pH 6.8

| | |
|---|---|
| KH₂PO₄ | 3.94 g |
| NaOH | adjust to a pH of 6.8 |
| Sodium Cholyltaurine | 5 mM |
| Lecithin | 1.5 mM |
| KCl | 16.4 g |
| Distilled water | sufficient |
| Total volume | 1000 mL |

### Medium D (FeSSIF, simulated intestinal fluid, fed condition):pH 5

| | |
|---|---|
| KH₂PO₄ | 8.65 g |
| NaOH | adjust to a pH of 5 |
| Sodium Cholyltaurine | 15 mM |
| Lecithin | 3.7 mM |
| KCl | 15.2 g |
| Distilled water | sufficient |
| Total volume | 1000 mL |

Medium A represents standard condition of gastric fluid, Medium B represents the standard condition of intestinal fluid; Medium C represents the simulated condition of intestinal fluid (fasted condition); Medium D represents the simulated condition of intestinal fluid (fed condition). In use, administration under fasted conditions means administration without ingestion of food for at least 8 hours. Administration under fed conditions means that food is ingested within 30 minutes before or after administration.

### EXAMPLE 6

Prescription (per tablet):
(1) Drug layer:

| | | |
|---|---|---|
| Tamsulosin hydrochloride | 0.2 | mg |
| Copovidone (Plasdone S630) | 80 | mg |
| Povidone (Plasdone K90) | 34 | mg |
| Yellow iron oxide | 0.07 | mg |
| Magnesium stearate | 1 | mg |
| Sillicon dioxide | 0.5 | mg |

(2) Push layer:

| | | |
|---|---|---|
| Sodium starch glycolate | 35 | mg |
| HPMC | 15 | mg |
| Carbomer | 6.3 | mg |
| Sodium chloride | 26 | mg |
| Copovidone (Plasdone S630) | 20 | mg |
| Red iron oxide | 1 | mg |
| Magnesium stearate | 0.5 | mg |
| Sillicon dioxide | 0.4 | mg |

(3) Semipermeability membrane:

| | | |
|---|---|---|
| Cellulose acetate | 19 | mg |

(4) Film coat:

| | | |
|---|---|---|
| Spectrablend ™ (CM 0165) | 18 | mg |

Preparation method and test method of drug release as example 5.

Results of the drug realease: The cumulated drug release at 1, 3, 5, 7, 12 and 16 hour is 12%, 34%, 50%, 67%, 90% and 95%.

### EXAMPLE 7

Preparation method as in Example 1 (without avoiding the light).

Prescription:
(1) Drug layer (per tablet):

| | | |
|---|---|---|
| Glipizide | 5 | mg |
| Povidone (Plasdone K90) | 20 | mg |
| Copovidone (Plasdone S630) | 69 | mg |
| Yellow iron oxide | 0.05 | mg |
| Magnesium stearate | 0.75 | mg |
| Silicon dioxide | 0.5 | mg |

| | |
|---|---|
| Alcohol | as required |

(2) Push layer (per tablet):

| | | |
|---|---|---|
| Sodium starch glycolate | 30 | mg |
| HPMC (K15M) | 14 | mg |
| carbomer (971PNF) | 10 | mg |
| Sodium chloride | 30 | mg |
| Copovidone (Plasdone S630) | 15 | mg |
| Red iron oxide | 0.95 | mg |
| Magnesium stearate | 0.48 | mg |
| Silicon dioxide | 0.5 | mg |

| | |
|---|---|
| Alcohol | as required |

(3) Semipermeability membrane coating solution contains cellulose
acetate, diethyl phthalate; then coating with moisture-proof film coat.

The drug release test results of the sample from Example 7 and a commercial product (trade name: Glucotrol XL, made by American Pfizer Inc.) show that both meet the requirement of effective drug release over a 24 hour period. But compared with mean drug release 0.89% of the commercial product at 2 hours, the test product is 6.00%. The test product has lower time lag. Compared with mean drug release 101.54% of the commercial product at 16 hour, the test product is 102.55%. The test product is more fully released.

### EXAMPLE 8

Under the condition of different pH value mediums, the drug release of the commercial tablet and tablet from Example 7 are determined separately. The results are shown in Table 2.

(1) Hydrochloric acid solution with 0.5% sodium dodecylsulfate (pH=1.2);

(2) Acetic acid-sodium acetate buffer solution with 0.5% sodium dodecylsulfate (pH=4.5);

(3) Simulated intestinal fluid without pancreatin (pH6.8).

TABLE 2
The drug release of glipizide (n = 6)

| Drug release | | Drug release (%) | | | | | | |
|---|---|---|---|---|---|---|---|---|
| medium | | 2 h | 4 h | 6 h | 8 h | 10 h | 12 h | 16 h |
| Own | pH 1.2 | 3.99 | 22.14 | 40.49 | 59.45 | 76.89 | 93.21 | 100.85 |
| product | pH 4.5 | 6.43 | 24.02 | 43.56 | 62.78 | 79.76 | 95.04 | 103.56 |
| | pH 6.8 | 7.59 | 23.61 | 41.39 | 60.17 | 78.63 | 93.04 | 103.23 |
| On-sale | pH 1.2 | 0.56 | 17.48 | 35.25 | 53.22 | 71.52 | 87.51 | 98.26 |
| product | pH 4.5 | 1.55 | 19.21 | 37.34 | 56.63 | 73.95 | 92.02 | 103.49 |
| | pH 6.8 | 0.56 | 19.55 | 37.95 | 57.09 | 75.51 | 92.68 | 102.86 |

The drug release of the test product from Example 7 and the commercial product (trade name: Glucotrol XL, made by American Pfizer Inc.) both meet the requirement of effective drug release over a 24-hour period. But compared to the commercial product, the test product has a less time lag and is more fully released.

### EXAMPLE 9

Preparation method as Example 1 (without avoiding the light).

Prescription:
(1) Drug layer (per tablet):

| | | |
|---|---|---|
| Glipizide | 5 | mg |
| Povidone (Plasdone K90) | 75 | mg |
| Yellow iron oxide | 0.05 | mg |
| Magnesium stearate | 0.75 | mg |
| Silicon dioxeide | 0.5 | mg |

| | |
|---|---|
| Alcohol | as required |

(2) Push layer (per tablet):

| | | |
|---|---|---|
| Croscarmellose sodium | 150 | mg |
| HPMC (K15M) | 5 | mg |
| Carbomer (971PNF) | 20 | mg |
| Sodium chloride | 35 | mg |
| Copovidone (Plasdone S630) | 25 | mg |
| Red iron oxide | 0.95 | mg |
| Magnesium stearate | 0.48 | mg |
| Silicon dioxide | 0.5 | mg |

| | |
|---|---|
| Alcohol | as required |

(3) Semipermeability membrane coating solution contains cellulose
acetate, diethyl phthalate; then coating with moisture-proof film coat.

### EXAMPLE 10

Studies of pharmacokinetics and bioavailability of formulations prepared according to the present invention compared to formulations marketed are carried in healthy volunteers.

Method: Randomized according to body weight index, 2-period, crossover study under fasted state. 24 healthy male volunteers were crossed and randomized to administer a single-dose of Glipizide controlled-release tablet (test drug, prepared according to the present invention) and Glucotrol® XL (reference drug, marketed) respectively. The blood concentrations were determined by method of LC-MS/MS, and pharmacokinetic parameters of two formulations and relative bioavailability of test drug were calculated with the software of 3P97, emphasizing on the difference of two drugs' blood concentrations during the initiatory period after administration, which means the difference of time lag of drug release in vivo.

Method in detail: 24 healthy male volunteers randomized to 2 groups, 12 volunteers per group, who were fasting after the supper of the day before study and were allowed to take food 2 hours later after administration, were crossed to orally administer 5 mg of test drug and reference drug respectively. They took standard food for lunch and were allowed to take some water during the study. 4 ml of blood sampling for each volunteer was done pre-dose and at 0.5, 1.0, 2.0, 3.0, 4.0, 6.0, 8.0, 10.0, 12.0, 16.0, 24, 36, and 48 hours after dosing. The samples were centrifuged and placed under −60° C. condition for determination.

After checking the data, blood concentrations of each time point were calculated with the weighted regression method and then data were collected with EXCEL software. Software of 3P97 was used to calculate the pharmacokinetic parameters.

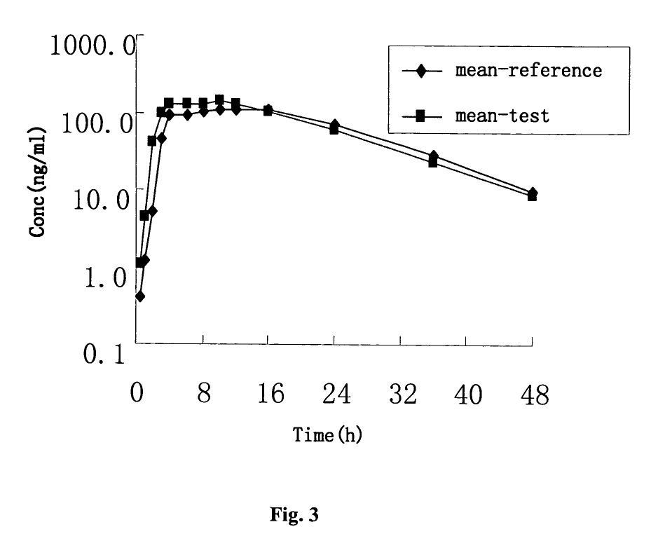

The curve of mean blood concentration to time is shown in FIG. 2. The data is shown in log scale in FIG. 3. The changing trends of two groups of curves are seen to be basically consistent. The test formulation and marketed formulation both show the excellent properties of controlled release. However, the initial blood concentration after administration of test formulation is obviously higher than that of the reference formulation, and the peak time of test formulation is shorter than that of reference formulation. Tₘₐₓ of the marketed formulation is 10.67±5.13 h, while that of test formulation is 9.08±2.76 h, which shows that time lag of drug release of test formulation is obviously shorter than that of marketed reference formulation.

### EXAMPLE 11

Preparation method as in Example 1 (without avoiding the light).

Prescription:
(1) Drug layer (per tablet):

| | | |
|---|---|---|
| Theophylline | 100 | mg |
| Povidone(Plasdone K-90D) | 5 | mg |
| Copovidone (Plasdone S630) | 23 | mg |
| Magnesium stearate | 1.5 | mg |
| Silicon dioxide | 0.5 | mg |

(2) Push layer (per tablet):

| | | |
|---|---|---|
| Sodium starch glycolate | 45 | mg |
| HPMC (K4M) | 30 | mg |
| HPC | 35 | mg |
| NaCl | 12 | mg |
| Copovidone (Plasdone S630) | 20 | mg |
| Red iron oxide | 1.1 | mg |
| Magnesium stearate | 0.6 | mg |
| Silicon dioxide | 0.4 | mg |

(3) Semipermeability membrane coating solution contains cellulose
acetate, diethyl phthalate; then coat with moisture-proof film coat.

### EXAMPLE 12

Preparation method as in Example 1 (without avoiding the light).

Prescription:
(1) Drug layer (per tablet):

| | | |
|---|---|---|
| Phenoxybenzamine Hydrochloride | 40 | mg |
| Povidone (Plasdone K-30) | 46 | mg |
| Copovidone (Plasdone S630) | 51 | mg |
| Magnesium stearate | 1.5 | mg |
| Silicon dioxide | 0.5 | mg |

(2) Push layer (per tablet):

| | | |
|---|---|---|
| Sodium starch glycolate | 35 | mg |
| HPMC (K100M) | 20 | mg |
| HPC | 35 | mg |
| NaCl | 24 | mg |
| Carbomer | 33 | mg |
| Red iron oxide | 1.1 | mg |
| Magnesium stearate | 0.6 | mg |
| Silicon dioxide | 0.4 | mg |

(3) Semipermeability membrane coating solution contains cellulose
acetate, diethyl phthalate; then coat with moisture-proof film coat.

### EXAMPLE 13

Preparation method as Example 1 (without avoiding the light).

Prescription:
(1) Drug layer (per tablet):

| | | |
|---|---|---|
| Carbimazole | 30 | mg |
| Povidone (Plasdone K-60) | 33 | mg |
| Copovidone (Plasdone S630) | 62 | mg |
| Magnesium stearate | 1.5 | mg |
| Silicon dioxide | 0.5 | mg |

(2) Push layer (per tablet):

| | | |
|---|---|---|
| Sodium starch glycolate | 35 | mg |
| HPMC (K100M) | 24 | mg |
| HPC | 41 | mg |
| NaCl | 18 | mg |
| Carbomer | 26 | mg |
| Red iron oxide | 1.1 | mg |
| Magnesium stearate | 0.6 | mg |
| Silicon dioxide | 0.4 | mg |

(3) Semipermeability membrane coating solution contains cellulose
acetate, diethyl phthalate; then coat with moisture-proof film coat.

### EXAMPLE 14

Preparation method as in Example 1 (without avoiding the light).

Prescription:
(1) Drug layer (per tablet):

| | | |
|---|---|---|
| Chlordiazepoxide | 20 | mg |
| Povidone (Plasdone K-60) | 54 | mg |
| Copovidone (Plasdone S630) | 51 | mg |
| Magnesium stearate | 1.5 | mg |
| Silicon dioxide | 0.5 | mg |

(2) Push layer (per tablet):

| | | |
|---|---|---|
| Sodium starch glycolate | 25 | mg |
| HPMC (K15M) | 30 | mg |
| HPC | 31 | mg |
| NaCl | 31 | mg |
| Carbomer | 29 | mg |
| Red iron oxide | 1.1 | mg |
| Magnesium stearate | 0.6 | mg |
| Silicon dioxide | 0.4 | mg |

(3) Semipermeability membrane coating solution contains cellulose
acetate, diethyl phthalate; then coat with moisture-proof film coat.

Test:

Under the condition of different pH value, the drug release test results of samples from Example 1 and the commercial product show that both meet the requirement of effective drug release over a 24-hour period. But compared with mean drug release 8.73% of the commercial product at 4 hours, the test product is 9.93%. The test product has less time lag. Compared with the mean drug release 95.8% of the commercial product at 24 hours, the test product is 99.0%. The test product is more fully released.

Drug release results of the samples from Example 5: The cumulated drug release at 1, 3, 5, 7, 12 and 16 hours is 10%, 33%, 53%, 76%, 93% and 96%. Drug release results of the samples from Example 6: The cumulated drug release at 1, 3, 5, 7, 12 and 16 hours is 12%, 34%, 50%, 67%, 90% and 95%. These data show that tablets from Example 5 and Example 6 can obtain a controlled release effect.

What is claimed is:

1. An osmotic pump device for administration of a low solubility biologically active substance at a controlled rate into a biological environment comprising
a core comprising a first layer containing a pharmaceutically effective amount of said low solubility biologically active substance and about 10 to 99 percent by weight of said first layer of hydrophilic polymer carrier, and a second layer comprising about 10 to 80 percent by weight of said second layer of water-insoluble volume-swellable polymers, about 80 to 10 percent by weight of said second layer of water-soluble osmopolymers, and about 5 to 50 percent by weight of said second layer of osmagents.

2. The device according to claim 1 wherein said low solubility biologically active substance has a solubility in water of no greater than about 10 mg/ml.

3. The device according to claim 1 wherein the ratio of weight of said first layer to said second layer is in the range of about 1:0.5 to 1:3.

4. The device according to claim 1 wherein said hydrophilic polymer carrier comprises polyvinylpyrrolidone polymers and/or copolymers.

5. The device according to claim 4 wherein the said polyvinylpyrrolidone polymer comprises a homopolymer of linear 1-vinyl-2-pyrrolidone groups.

6. The device according to claim 4 wherein said polyvinylpyrrolidone copolymer comprises a copolymer of 1-vinyl-2-pyrrolidone and vinyl acetate in the mass proportion of about 1:10 to 10:1.

7. The device according to claim 1 wherein said osmopolyers in said second layer are selected from the group consisting of acrylic acid polymers, acrylic acid copolyers, hydroxypropylmethyl cellulose, polyvinypyrrolidone polymers, polyvinylpyrrolidone copolymer and mixtures of two or more thereof.

8. The device according to claim 7 wherein the said acrylic acid polymers and/or copolymers comprise homopolymers of acrylic acid, crosslinked with an allyl ether pentaerythritol, allyl ether of sucrose, or allyl ether of propylene.

9. A device according to claim 1 wherein said water-insoluble volume-swellable polymers in said second layer are selected from the group consisting of sodium starch glycolate, low-substituted hydroxypropyl cellulose, crosslinked carboxylmethyl cellulose sodium and mixtures of two or more thereof.

10. The device according to claim 1 wherein osmagents in said second layer are selected from the group consisting of water soluble inorganic salts, organic acids, saccharides and mixtures of two or more thereof.

11. The device according to claim 10 wherein said salts are selected from the group consisting of sodium chloride, potassium chloride, magnesium chloride, potassium sulphate, sodium sulphate and magnesium sulphate.

12. The device according to claim 10 wherein said acids are selected from the group consisting of ascorbic acid and tartaric acid.

13. The device according to claim 10 wherein said saccharides are selected from the group consisting of mannitol, sorbitol, xylitol, glucose and sucrose.

14. The device according to claim 1 wherein said first and second layers each further comprise a lubricant, a glidant, and a colorant.

15. The device according to claim 14 wherein said lubricant and/or glidant is selected from the group consisting of magnesium stearic acid, silicon dioxide and mixtures of two or more thereof.

16. The device according to claim 1 further comprising a wall surrounding said core comprising a semi-permeable material permeable to the passage of an exterior fluid and substantially impermeable to the passing of said biologically active substance.

17. The device according to claim 16 wherein said semi-permeable material comprises cellulose polymers.

18. A device according to claim 17 wherein said cellulose polymers comprise cellulose ethers, cellulose esters or cellulose ester ethers.

19. The device according to claim 16 further comprising a passageway in said wall communicating with said first layer and the exterior of said device for delivery of said biologically active substance from said device.

20. A device according to claim 19 wherein said passageway is of the diameter of about 0.2 to 1.2 mm.

21. A device according to claim 4 wherein said polyvinylpyrrolidone polymers and/or copolymers in said first layer have an average molecular weight in the range of about 5000 to 3,000,000.

22. A method of preparation of said core of said device according to claim 1 comprising:
preparing the composition for said first layer by
a) separately passing biologically active substance, said hydrophilic polymer carrier and optional ingredients desired for said first layer through a 60-mesh sieve;
b) mixing said biologically active substance with said hydrophilic polymer carrier and said optional ingredients for said first layer to form a first ingredient mixture;
c) spraying said first ingredient mixture with an alcohol solution to form a first wet mixture;
d) granulating said first wet mixture;
and preparing, said osmagents and optional ingredients desired for said second layer through a 60-mesh sieve;
ii) mixing said osmopolymers, said water-insoluble water-swellable polymers, said osmagents and said optional ingredients for said second layer to form a second ingredient mixture;
iii) spraying said second ingredient mixture with alcohol solution to form a second wet mixture;
iv) granulating said second wet mixture;
1) pressing at least a portion of said granulated first wet mixture from step (d) to form said first layer;
2) applying at least a portion of said granulated second wet mixture from step (iv) onto said first layer from step (1) to form a core precursor;
3) pressing said core precursor to form said core comprising said first and second layers.

23. The method according to claim 22 wherein said first layer contains magnesium stearic acid, colorants, polyvinylpyrrolidone polymers and/or copolymers, osmagents and/or silicon dioxide and said second layer contains adhesives, colorants and/or glidants.

24. The method according to claim 22 or 23 further comprising the step 4) of coating said core with a semi-permeable membrane.

25. The method according to claim 24 further comprising the step 5) forming a passageway in said semi-permeable membrane connecting said first layer with the exterior of said device for release of said biologically active substance.

26. The method according to claim 25 wherein the diameter of said passageway is in the range of about is 0.2 to 1.2 mm.

27. The method according to claim 24 further comprising the step of applying an anti-damp film over said semi-permeable membrane.
